(19) United States
(12) Patent Application Publication
(10) Pub. No.: US 2011/0306942 A1
(43) Pub. Date: Dec. 15, 2011

(54) FINGER APPLICATOR DEVICE

(76) Inventor: Gerald W. THORPE, Yucaipa, CA (US)

(21) Appl. No.: 12/813,769

(22) Filed: Jun. 11, 2010

(51) Int. Cl. A61M 35/00

(52) U.S. Cl. 604/289

(57) ABSTRACT

A dosage pad for an applicator includes an absorbent matrix capable of retaining a product, a protective covering for sealing the product in the absorbent matrix, a means for removing the protective covering and an attachment means. The protective covering can be suitably removed from the absorbent matrix by the provision of a tab or a cord which exposes the absorbent matrix upon exertion of force. Absorbed in the absorbent matrix is a quantity of product which may include a medicament such as a cream or ointment. Additionally, the dosage pad may be optionally affixed to a glove to allow for digital application of a product. Either a single or multiple dosage pads may be affixed to the glove by means of an adhesive layer or fusion. The size of the dosage pads may also vary depending on the specific treatment site upon which application of a product is desired.

### TECHNICAL FIELD

The present invention relates generally to a finger applicator for applying a product. More specifically, the present invention relates to a finger applicator that can be affixed or provided integral to a glove for more effectively applying a product to a patient.

### BACKGROUND OF THE INVENTION

Numerous medicaments in cream, ointment and liquid bases have been formulated for topical application. In many circumstances, these substances must be carefully communicated to a treatment site in order to avoid contamination of surrounding areas.

In some instances, the application of a medicament for topical use is accomplished directly with a finger applicator. However, where a bare finger is used, the approach is both unsanitary and inefficacious. Not only is there a medicament residue that has to be cleansed from the finger but, in addition, since the surface of the finger harbors microorganisms, an opportunity is presented for harmful contamination. Thus, utilization of this dispensing approach requires assiduous finger cleaning both prior to and after dispensing.

Alternatively, in instances where a covered finger applicator such as a finger cot are used, medicament is applied to the finger applicator and thereto the treatment site. Unfortunately, if the treatment site is difficult to see, e.g. gingival or other mucosa inside the mouth, areas within the vagina or areas such as the prostate and rectum, it is difficult to accurately dispense the medicament at the treatment site. Furthermore, the amount of medicament retained on the finger applicator is often excessive and subject to slippage from the finger applicator, particularly when medicament is being applied to an area which is difficult to see. As a result, the medicament will often slip off of the finger applicator prior to being applied to the treatment site. Further, when applying medicament to treatment sites which are difficult to see, the subject applying the medicament does not know the precise amount of medicament that actually reaches the treatment site, because much of the medicament is rubbed off the finger applicator. This can result in either an overdose or underdose of medication to the treatment site in addition to unintended application of medicament to unaffected areas which can cause irritation or more severe adverse effects. Thus, utilization of this dispensing approach leads to either excessive or insufficient dispensing of medicament to the treatment site as well as potential application of medicament to unaffected areas prior to reaching the treatment site.

Furthermore, in instances where an applicator is provided with an absorbent material on the distal end to aid in dispensing medicament, the medicament is often applied either just prior to application or pre-applied to the absorbent material. However, in many instances, a number of medicaments may undergo potential adverse effects from exposure to atmospheric elements such as water vapor, light, oxygen or other gases. Exposure to such atmospheric elements may affect the decomposition rate of the medicament, the dissolution rate of the active medicament or the effectiveness of the active element in the medicament. Thus, depending on the type of medicament being applied to the treatment site, it may be essential to provide an effective seal for the medicament prior to application.

Accordingly, there is a need to address these and other shortcomings in the art as detailed below.

### SUMMARY OF THE INVENTION

The present disclosure relates to a finger applicator for the application of a product. The finger applicator comprises a finger sheath having a proximal end and a distal end, a dosage pad comprised of an absorbent matrix and a removable protective covering provided on the absorbent matrix for sealing the product in the absorbent matrix, wherein the dosage pad is attached to the distal end of the finger sheath.

In one of various aspects of this disclosure, the absorbent matrix is comprised of an absorbent material capable of retaining a product, which may be a cotton fabric, rayon-cotton fabric, foam or textile material, natural or artificial sponge, polymeric product, cellulose product or gauze.

In yet another aspect of this disclosure, the protective covering provided on the absorbent matrix is comprised of a material suitable for sealing a product in the absorbent matrix such as polypropylene, polysterene, polyamide, polyvinyl chloride, polyethylene, silicone treated paper or a polymeric film.

In still another aspect of this disclosure, the protective covering can be removed by pulling a cord affixed within the protective covering such that when force is applied to the cord a fissure is created along the protective covering creating an opening for releasing the product.

In yet another aspect of this disclosure, the dosage pad is attached to the distal end of the finger applicator with an adhesive, which may be an adhesive comprised of a polyvinyl acetate, polyimide, silicone, acrylic or epoxy composition.

Another aspect of this disclosure is a glove which has at least one finger sheath. The glove may be comprised of latex but may additionally be comprised of vinyl, nitrile or a synthetic material and so forth. An additional aspect of this disclosure also includes a glove wherein a single or multiple dosage pads are fused integral to the glove.

Numerous other advantages and features of the described embodiments will become readily apparent from the detailed description of preferred embodiments of the invention, from the claims and from accompanying drawings in which like numerals are employed to designate like parts throughout the same.

### BRIEF DESCRIPTION OF THE DRAWINGS

FIG. 1a illustrates a perspective view of a first embodiment of a finger applicator.

FIGS. 1b, 1c and 1d illustrate alternative embodiments of the invention.

FIG. 2 illustrates an embodiment of a glove with a dosage pad affixed to a finger applicator.

FIG. 3 illustrates an alternative embodiment of a glove with a dosage pad to a finger applicator.

FIGS. 4a and 4b illustrate other various embodiments of the invention.

### DETAILED DESCRIPTION OF THE DRAWINGS

The disclosure provided below relates generally to a finger applicator for applying a product, more specifically, to a finger applicator comprising a finger sheath and a dosage pad attached to the finger sheath, wherein the dosage pad can be affixed or provided integral to the finger sheath, for more effectively applying product to a patient. While the various embodiments are shown in the drawings, it should be understood, however, that they are to be considered an exemplification of the principles of the invention and are not intended to limit the spirit and scope of the subject matter described herein and/or claims of the embodiments illustrated.

Further, unless defined otherwise, all terms used herein have the same meaning as are commonly understood by one of skill in the art to which this invention belongs. All patents, patent applications and publications referred to throughout the disclosure herein are incorporated by reference in their entirety.

The term “applicator” refers to any instrument capable of being used to apply a product such as, but not limited to, a digital applicator such as a hand or glove and a swab.

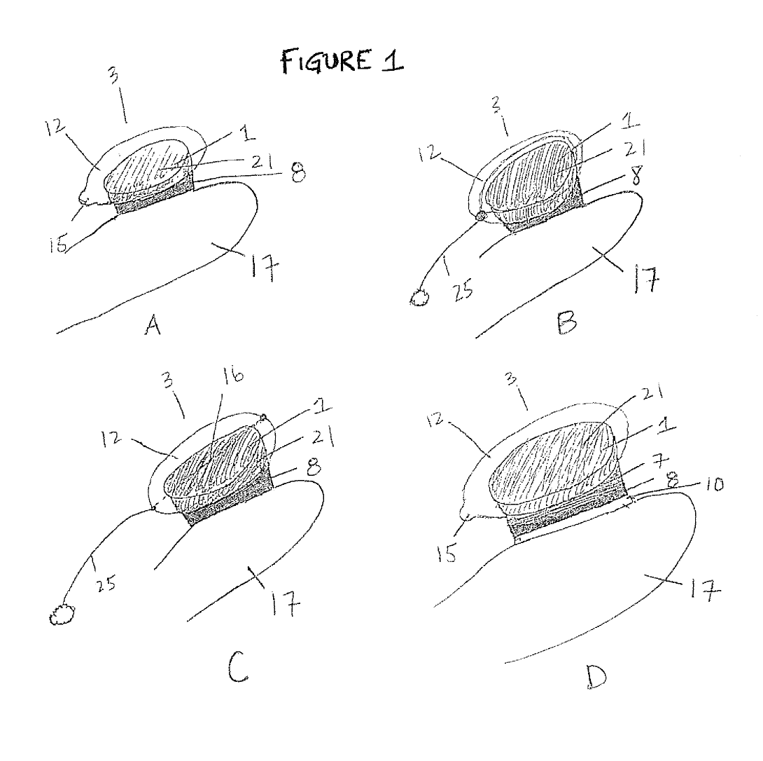

Referring now in detail to the drawings, FIG. 1a illustrates an exemplary finger applicator comprising a finger sheath 17 with a dosage pad 3 attached. The dosage pad 3 comprises an absorbent matrix 1 capable of retaining a product 21. The absorbent matrix 1 is conventionally constructed and can be made of any suitable absorbent material, such as a sponge, that is capable of holding volumes of a product 21 therein. Further examples of suitable materials which the absorbent matrix 1 may be made of include, but are not limited to, cotton fabrics, rayon-cotton fabrics, foam or textile materials, natural and artificial sponges, polymeric products, cellulose products and gauze. The absorbent matrix 1 can have a circular or elliptical shape but may be constructed in any desired shape. In addition, the absorbent matrix 1 can have a substantially consistent thickness to provide a flattened shape for ease of application. The absorbent matrix 1 may be made available in any variation of sizes to accommodate both the size of the applicator or the quantity of product 21 to be applied. Additionally, the selected product 21 to be applied to a treatment site may govern the selection of a suitable material to construct the absorbent matrix 1. One skilled in the art would recognize that factors such as absorbability and rate of product transmission to a treatment site or other factors would be important in the selection of a suitable material to construct the absorbent matrix 1. Furthermore, as shown in FIG. 1d, when the dosage pad 3 is being affixed to an applicator, a backing layer 7 may be provided on the bottom surface of the absorbent matrix 1 to provide stability and inclusion of an adhesive layer 8.

The absorbent matrix 1 is enclosed by a protective covering 12 so as to effectively hermetically seal the absorbent matrix and/or any product 21 contained in the absorbent matrix 1 from exposure to agents that would interfere with the effectiveness of the product 21. Agents may include atmospheric elements such as air, water vapor or potentially contaminating areas surrounding the treatment site. The protective covering 12 can be made of any suitable material known to one skilled in the art such as a plastic e.g. polypropylene, polysterene, polyamide, polyvinyl chloride and polyethylenes, silicone treated paper or a polymeric film and so forth. The protective covering 12 can be hermetically sealed with respect to the product 21 so as to seal and isolate the product 21 within the absorbent matrix 1. In circumstances where it is desired to have an absorbent matrix 1 of substantial thickness the protective covering 12 may be a cap. Alternatively, where the absorbent matrix 1 is desired to be of a thin composition the protective covering 12 may be a film or sheet sufficiently secure to maintain the hermetic seal over the absorbent matrix 1 prior to application. The protective covering 12 may be adhered around the absorbent matrix 1 by any method sufficient to preserve the hermetic seal of the absorbent matrix 1 when the protective covering 12 is being removed. For example, the protective covering 12 may be adhered around the absorbent matrix 1 with an adhesive or by heat-sealing, for example.

Removal of the protective covering 12 may be accomplished by any means sufficient to expose the absorbent matrix 1 prior to application. For example, as shown in FIG. 1a, the protective covering 12 may include a tab 15 as a means for removing the protective covering 12. Exerting sufficient force on the tab 15 will remove the protective covering 12 and expose the absorbent matrix 1 prior to application. The tab 15 can be made of the same material as the protective covering 12 or any other suitable material known in the art. Alternatively, as shown in FIG. 1b, a cord 25 may be utilized in place of a tab 15 as the means for removing the protective covering 12. The cord 25 may be comprised of thread, plastic or any suitable material sufficient to allow for removal of the protective covering upon applying force on the cord 25. The composition of the cord 25 may depend on the method in which the protective covering 12 is attached to the absorbent matrix 1. In one embodiment, as shown in FIG. 1b, the cord 25 is interwoven along the perimeter of the protective covering 12 to provide sufficient gripping such that when adequate force is applied on the cord 25 the protective covering 12 is removed, thus exposing the absorbent matrix 1 to allow for application of a product 21 to a treatment site.

In another embodiment, as shown in FIG. 1c, the cord 25 is woven across the surface of the protective covering 12, which may be provided with a non-penetrative perforation 16 such that when adequate force is applied on the cord 25 a separation is created along the surface of the protective covering 12, thus exposing the absorbent matrix 1 to allow for application of a product 21 to a treatment site. In one embodiment, as shown in FIG. 1c, exerting force on the cord 25 will create a separation across the surface of the protective covering 12. Subsequent force exerted with the exposed dosage pad 3 pressing against the treatment site will cause the remainder of the protective covering 12 to collapse and allow the absorbent matrix 1 to deliver product 21 to the treatment site. In instances where the treatment site is difficult to observe, the length of the cord 25 may be extended sufficiently to allow for removal of the protective covering 12 away from the unobservable treatment site after the dosage pad 3 has been directed to the treatment site. Extension of the cord 25 allows for removal of the protective covering 12 when the applicator is directed to areas such as mucosa or a cervix, for example.

On the bottom surface of the absorbent matrix 1, the dosage pad 3 is provided with an adhesive layer 8. In another embodiment, the adhesive layer 8 may be attached to a backing layer 7 on the bottom surface of the absorbent matrix 1. In some embodiments, the adhesive layer 8 can be a permanent, water-resistant, hypoallergenic adhesive which adheres more strongly to the dosage pad 3 than to the applicator. Additionally, the adhesive layer 8 can be made of any suitable material some examples beings polyvinyl acetate, polyimide, silicone compositions, acrylics and epoxies, and so forth. The adhesive layer 8 can also be in the form of a film which can cover the entire bottom surface of the absorbent matrix 1 or the backing layer 7. Further, the adhesive layer 8 can be in the form of a pattern of points, a series of bands, or may extend only in the central region of the absorbent matrix 1 or the backing layer 7. Where an adhesive layer 8 is provided on the dosage pad 3 a protective layering 10 may be included on the adhesive layer 8 such as a film or strip to preserve the adhesive qualities of the adhesive layer 8 prior to use. In this embodiment, the protective layering 10 would be removed just prior to affixing the dosage pad 3 to an applicator.

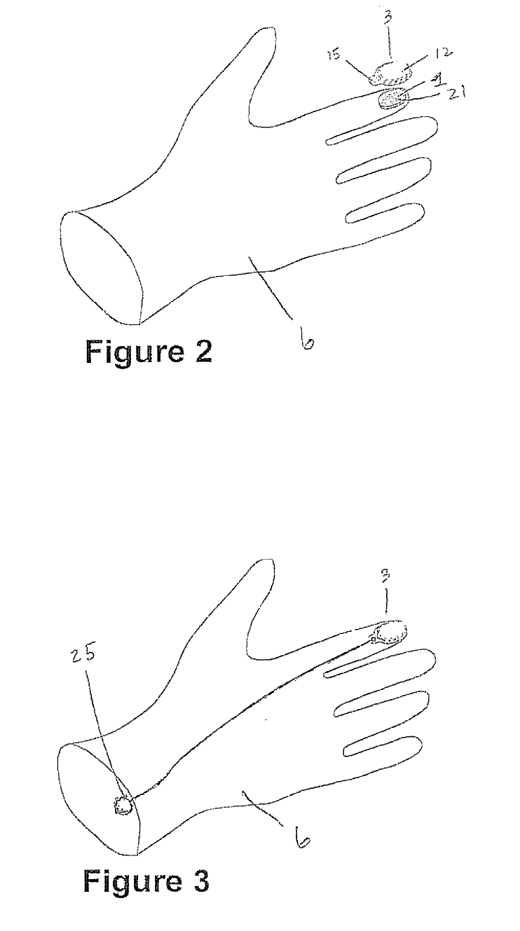

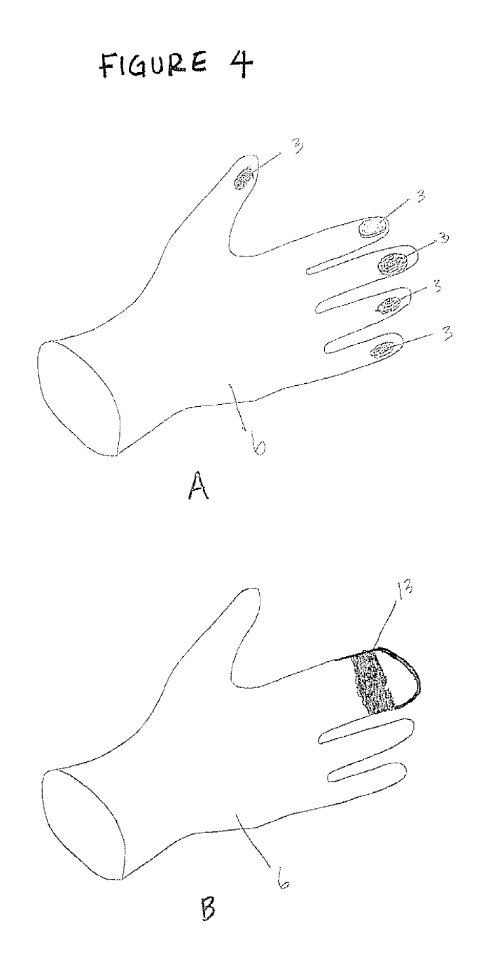

As shown in FIG. 2, a finger sheath 17 can be affixed or integrally fused to a finger on a glove 6. In some embodiments, as further shown in FIG. 2, the finger sheath 17 may be fused integral to the glove 6 to provide a glove of single and unitary construction. Alternatively, the finger sheath 17 may also be provided over a finger on the glove. The dosage pad 3 provided on the finger sheath 17 provides protection for a finger and/or hand from the product 21 being applied to a treatment site, the affected treatment site or surrounding areas. Additionally, as shown in FIGS. 4 and 4b, multiple finger sheaths may be affixed or integrally fused to a plurality of fingers on the glove including across multiple fingers such as an index and middle finger or an index finger and thumb. One of ordinary skill in the art will understand that numerous other embodiments can be achieved by placing a single or multiple finger sheaths in one or more positions on the glove 6.

In one embodiment, the glove 6 is a conventional latex glove. The glove 6 may alternatively be comprised of vinyl, nitrile, synthetic or any other suitable material known by one of ordinary skill in the art.

Once a finger sheath 17 has been affixed to an applicator such as a glove 6, the applicator is directed to a desired treatment site. In instances wherein the positioning of the applicator cannot be observed, such as wherein the treatment site comprises areas unobservable for self application such as mucosa or areas within the vagina or rectum, tactile sensory perception is utilized to properly position the applicator by feel. After the treatment site has been located, the protective covering 12 is removed to expose the absorbent matrix 1. Thereafter, compressive force is exerted toward the treatment site with the absorbent matrix 1 pressing against the treatment site sufficiently to release the product 21. The requisite motion against the treatment site and/or the time which the absorbent matrix 1 must be pressed against the treatment site may depend on the product 21 being applied.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

With respect to the above description, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention. It should also be appreciated that the dosage pad of the present invention is suitable for usage in the application of various products and/or preparations and is not limited in its utility to the application of medicaments. For example, the dosage pad is well suited for storing and applying cosmetics. Furthermore, it should also be appreciated that the dosage pad of the present invention may be provided with an absorbent matrix which has not been preloaded with a product or medicament in the event one desires to load the product or medicament following removal of the protective covering.

It is to be understood that the invention is capable of other embodiments and of being practiced and carried out in various ways. Also, is to be understood that the invention is not limited in its application to the details of construction and that all matter herein described or shown in the accompanying drawings is to be interpreted as illustrative and not limiting.

What is claimed is:

1. A finger applicator for the application of a product comprising:
a finger sheath having a proximal end and a distal end;
a dosage pad comprised of an absorbent matrix containing a product; and
a removable protective covering provided on the absorbent matrix for sealing the product in the absorbent matrix;
wherein the dosage pad is attached to the distal end of the finger sheath.

2. The finger applicator of claim 1, wherein the absorbent matrix is comprised of a cotton fabric, rayon-cotton fabric, foam or textile material, natural or artificial sponge, polymeric product, cellulose product or gauze.

3. The finger applicator of claim 1, wherein the protective covering is comprised of polypropylene, polysterene, polyamide, polyvinyl chloride, polyethylene, silicone treated paper or a polymeric film.

4. The finger applicator of claim 1, wherein the protective covering is removed by pulling a cord affixed within the protective covering such that upon applying force to the cord a fissure is created along the protective covering creating an opening for releasing the product.

5. The finger applicator of claim 1, wherein the dosage pad is attached to the distal end of the finger sheath with an adhesive.

6. The finger applicator of claim 5, wherein the adhesive is comprised of a polyvinyl acetate, polyimide, silicone, acrylic or epoxy composition.

7. A glove having at least one finger sheath according to claim 1.

8. The glove of claim 7, wherein the glove is comprised of latex, vinyl, nitrile, or synthetic material.

9. The glove of claim 7, wherein the dosage pad of is fused integral to the glove.

10. The glove of claim 7, wherein a plurality of dosage pads are affixed to the glove by an adhesive sufficient to maintain the dosage pad to the glove during application.

11. The glove of claim 7, wherein a plurality of dosage pads are fused integral to the glove.
